(19) United States
(12) Patent Application Publication
(10) Pub. No.: US 2025/0195148 A1
(43) Pub. Date: Jun. 19, 2025

(54) METHOD AND SYSTEM FOR SEQUENCE-AWARE ESTIMATION OF ULTRASOUND PROBE POSE IN LAPAROSCOPIC ULTRASOUND PROCEDURES

(71) Applicant: EDDA TECHNOLOGY, INC., Princeton, NJ (US)

(72) Inventors: Xiao Qi, Edison, NJ (US); Guo-Qing Wei, Plainsboro, NJ (US); Li Fan, Belle Mead, NJ (US); Xiaolan Zeng, Princeton, NJ (US); Jianzhong Qian, Princeton Junction, NJ (US)

(21) Appl. No.: 18/541,792

(22) Filed: Dec. 15, 2023

(51) Int. Cl. A61B 34/20; A61B 34/10; G06T 7/73; G16H 30/40

(52) U.S. Cl. CPC A61B 34/20; A61B 34/10; G06T 7/75; G16H 30/40; A61B 2034/105; A61B 2034/107; A61B 2034/2065; G06T 2207/10136; G06T 2207/20081; G06T 2207/20124; G06T 2207/30196

(57) ABSTRACT

The present teaching relates to estimating 3D pose of an ultrasound probe. An ultrasound image is acquired by an ultrasound probe deployed at a three-dimensional (3D) probe pose during a medical procedure directed to a target organ. A mask for each two-dimensional (2D) anatomical structure is identified and a corresponding label is estimated from the ultrasound image to generate an ASM/label pair. If a sequence of prior 3D probe poses does not exist, an ASM/label representation for the ultrasound image is generated based on the ASM/label pairs from the ultrasound image and used to estimate the 3D pose of the probe via an ASM-pose mapping model. If the sequence of prior 3D probe poses exists, the 3D probe pose is predicted based on the sequence of prior 3D probe poses and an ASM/label representation is accordingly generated based on a virtual ultrasound image created based on the predicted 3D probe pose.

### BACKGROUND

### 1. Technical Field

The present teaching generally relates to computers. More specifically, the present teaching relates to signal processing.

### 2. Technical Background

With the advancement of technologies, more and more tasks are now performed with the assistance of computers. Different industries have benefited from such technological advancement, including the medical industry, where large volume of image data, capturing anatomical information of a patient, may be processed by computers to identify anatomical structures of interest (e.g., organs, bones, blood vessels, or abnormal nodule), obtain measurements thereof (e.g., dimension of a nodule growing in an organ), and visualize relevant features (e.g., three-dimensional (3D) visualization of an abnormal nodule). Such information may be used for a wide variety of purposes. For example, a 3D model may be constructed for an organ (e.g., a liver in terms of its volume, shape, or size along different dimensions) or for different parts of the organ (e.g., a nodule growing inside a liver, vessel structures inside and near the organ). An example of a 3D model 100 for a liver is shown in FIG. 1A with a 3D vessel tree model 110 shown in FIG. 1B constructed based on vessels detected inside of the liver.

Such constructed 3D models may be used for different purposes, including diagnosis, presurgical planning, and in-surgical navigation, including in a laparoscopic ultrasound (LUS) procedure. FIG. 1C shows a LUS procedure setting where a patient 130 on a surgical table 120 is undergoing a laparoscopic procedure. A laparoscopic camera 140 is inserted into the patient's body and can be moved to reach a position to capture video images 150 of a scene observable by the camera 140. While a user such as a surgeon is manipulating the surgical tool to perform a surgical action, the laparoscopic camera 140 may be moved to near the surgical tool to reveal what is near the surgical instrument, which may provide useful visual guidance to the surgeon. The laparoscopic camera 140 may acquire the visual information about the surface of the anatomical structures such as an organ, it cannot provide information beneath the surface. Thus, an ultrasound probe 160 may also be inserted into the patient's body during the LUS procedure and is used to reveal what is beneath the organ. For instance, in a liver resection surgery, prior to a surgeon to cut open the liver, an ultrasound probe may be used to help the surgeon to “see” what is inside of the liver in order to assist the surgeon to determine how to proceed to cut open the liver. An exemplary ultrasound image 170 acquired by an ultrasound probe 160 is illustrated in FIG. 1C.

As commonly known, an ultrasound probe operates by sending out a beam of sound waves, which get reflected to the ultrasound probe by object boundaries along the path of the beam and an ultrasound image is generated based on the reflected sound waves. In general, an ultrasound probe may operate with some parameters, e.g., it may “see” information on a plane of a certain depth determined based on such parameters. This is illustrated in FIG. 1D, where the ultrasound probe 160 scans an organ 180 and what is “observable” by the ultrasound is information on a plane 190. Because of that, what is captured by an ultrasound probe may be restrictive or incomplete, as shown in FIG. 1E, where an ultrasound image 170 shows information corresponding to a tumor 170-1 and some vessel structures 170-2 and 170-3 but such information is incomplete and does not capture the 3D structures of the corresponding anatomical structures. The reason is that what is captured by an ultrasound probe is limited to a plane (e.g., 190) reachable by the sound wave emitted by the ultrasound probe.

As compared with the full 3D vessel tree 110 of the organ, as shown in FIG. 1B, the structures 170-2 and 170-3 as observed in the ultrasound image 170 may correspond to some vessels in the vessel tree 110. Knowing which corresponding vessels (e.g., a portal vein or a hepatic vein) may provide effective assistance to a surgeon. For example, in a liver resection operation, certain blood vessels may need to be clamped to stop the blood flow before the resection. However, the partial information from an ultrasound image makes it difficult to determine.

Thus, there is a need for a solution that addresses the challenges discussed above.

### SUMMARY

The teachings disclosed herein relate to methods, systems, and programming for information management. More particularly, the present teaching relates to methods, systems, and programming related to hash table and storage management using the same.

In one example, a method, implemented on a machine having at least one processor, storage, and a communication platform capable of connecting to a network for estimating 3D pose of an ultrasound probe. An ultrasound image is acquired by an ultrasound probe deployed at a three-dimensional (3D) probe pose during a medical procedure directed to a target organ. A mask for each two-dimensional (2D) anatomical structure is identified and a corresponding label is estimated from the ultrasound image to generate an ASM/label pair. If a sequence of prior 3D probe poses does not exist, an ASM/label representation for the ultrasound image is generated based on the ASM/label pairs from the ultrasound image and used to estimate the 3D pose of the probe via an ASM-pose mapping model. If the sequence of prior 3D probe poses exists, the 3D probe pose is predicted based on the sequence of prior 3D probe poses and an ASM/label representation is accordingly generated based on a virtual ultrasound image created based on the predicted 3D probe pose.

In a different example, a system is disclosed for estimating 3D pose of an ultrasound probe. An ultrasound probe is deployed at a 3D probe pose to acquire an ultrasound image during a medical procedure directed to a target organ. A sequence-aware ultrasound probe pose estimator is provided to first identify each two-dimensional (2D) anatomical structure from the ultrasound image and estimate a label therefor to generate an ASM/label pair. If a sequence of prior 3D probe poses does not exist, an ASM/label representation for the ultrasound image is generated based on the ASM/label pairs from the ultrasound image and used to estimate the 3D pose of the probe via an ASM-pose mapping model. If the sequence of prior 3D probe poses exists, the 3D probe pose is predicted based on the sequence of prior 3D probe poses and an ASM/label representation is accordingly generated based on a virtual ultrasound image created based on the predicted 3D probe pose.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for estimating a 3D pose of an ultrasound probe and application thereof. The information, when read by the machine, causes the machine to perform the following steps. An ultrasound image is acquired by an ultrasound probe deployed at a three-dimensional (3D) probe pose during a medical procedure directed to a target organ. A mask for each two-dimensional (2D) anatomical structure is identified and a corresponding label is estimated from the ultrasound image to generate an ASM/label pair. If a sequence of prior 3D probe poses does not exist, an ASM/label representation for the ultrasound image is generated based on the ASM/label pairs from the ultrasound image and used to estimate the 3D pose of the probe via an ASM-pose mapping model. If the sequence of prior 3D probe poses exists, the 3D probe pose is predicted based on the sequence of prior 3D probe poses and an ASM/label representation is accordingly generated based on a virtual ultrasound image created based on the predicted 3D probe pose.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIGS. 1A-1B show an exemplary 3D model of an organ and 3D vessel tree model for vessels inside the organ;

FIG. 1C shows a LUS surgical setting;

FIGS. 1D-1E illustrate the operational characteristics of an ultrasound probe;

FIG. 2 depicts an exemplary high level system diagram of a 3D ultrasound probe pose (USPP) based anatomical structure (AS) visualization framework, in accordance with an embodiment of the present teaching;

FIG. 3A illustrates exemplary ultrasound probe poses selected as basis for generating a mapping model, in accordance with an embodiment of the present teaching;

FIGS. 3B-3C illustrate discrete anatomical structure mask (ASM)-pose mappings automatically generated based on selected probe poses, in accordance with different embodiments of the present teaching;

FIG. 3D illustrates an ASM-pose mapping model generated via machine learning based on discrete ASM-pose mapping training data, in accordance with an embodiment of the present teaching;

FIG. 3E shows an exemplary ASM identified from a 2D ultrasound image corresponding to some portion of a 3D vessel tree;

FIG. 4A is a flowchart of an exemplary pre-surgery process of generating an ASM-pose mapping model based on basis 3D US probe poses, in accordance with an embodiment of the present teaching;

FIG. 4B is a flowchart of an exemplary in-surgery process for estimating a 3D ultrasound probe pose in an LUS procedure based on an ASM-pose mapping model, in accordance with an embodiment of the present teaching;

FIG. 5A depicts an exemplary high level system diagram of an ASM-pose mapping model generator, in accordance with an embodiment of the present teaching;

FIG. 5B is a flowchart of an exemplary process of an ASM-pose mapping model generator, in accordance with an embodiment of the present teaching;

FIG. 6A depicts an exemplary high level system diagram of a pose-based 2D ASM determiner, in accordance with an embodiment of the present teaching;

FIG. 6B is a flowchart of an exemplary process of a pose-based 2D ASM determiner, in accordance with an embodiment of the present teaching;

FIG. 7A depicts an exemplary high level system diagram of a sequence-aware US pose estimator, in accordance with an embodiment of the present teaching;

FIG. 7B is a flowchart of an exemplary process of a sequence-aware US pose estimator, in accordance with an embodiment of the present teaching;

FIG. 8A is a flowchart of an exemplary process for enhancing ASM labeling based on an estimated 3D US pose, in accordance with an embodiment of the present teaching;

FIG. 8B is a flowchart of an exemplary process for improving an estimated 3D US pose based on enhanced ASM labels, in accordance with an embodiment of the present teaching;

FIG. 9 is an illustrative diagram of an exemplary mobile device architecture that may be used to realize a specialized system implementing the present teaching in accordance with various embodiments; and

FIG. 10 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well known methods, procedures, components, and/or system have been described at a relatively high-level, without detail, in order to avoid unnecessarily obscuring aspects of the present teachings.

The present teaching discloses exemplary methods, systems, and implementations for a 3D USPP based AS visualization framework in an LUS procedure. An LUS procedure has been used for different surgeries such as laparoscopic liver resection as the use of an ultrasound probe may provide views of anatomical structured beneath an organ such as vessels and tumors. Prior to a surgery, 3D organ models may be constructed based on 3D medical image data obtained from scans such as computerized tomography (CT) or magnetic resonance imaging (MRI). Ultrasound and CT registration during a surgery may be useful in revealing the corresponding 3D anatomical structures based on what is visible in an ultrasound image. In a LUS procedure, as images acquired by a laparoscopic camera have a limited field of view without being able to reveal adequate anatomical structural information, it is usually not possible to register with a 3D model. On the other hand, ultrasound images acquired via an ultrasound probe in the same procedure are generally quite noisy and partial information so that they also cannot provide adequate anatomical structure information to enable the registration with the 3D models.

The present teaching discloses a framework of estimating the 3D pose of an ultrasound probe based on what is visible in a 2D ultrasound image and then use such estimated 3D ultrasound probe pose to visualize 3D anatomical structures that correspond to what is partially seen in 2D images. To visualize, the estimated 3D probe pose is used to determine a corresponding perspective with respect to the 3D organ models so that 3D anatomical structures in the 3D organ models as viewed from the perspective can be visualized. Such a visualization enables a user such as a surgeon to “see” the 3D anatomical structures beneath the surface of an organ that correspond to what is visible in an 2D ultrasound image. To estimate an ultrasound 3D probe pose, the framework according to the present teaching involves operations ta both pre-surgical and in-surgical stages.

In a pre-surgical stage, various assumed 3D probe poses may be obtained, each of which may be used to create a 2D virtual ultrasound image with a perspective determined by the 3D probe pose. With respect to each of such 2D virtual ultrasound images, 2D anatomical structures may be identified and corresponding anatomical structure masks (ASMs) and an label associated therewith may be obtained. An ASM may correspond to a binarized image with pixels on an anatomical structure being in one state (e.g., state 0) and everywhere else being in another state (e.g., state 1). A label associated with an ASM may be determined from the 3D model. Based on a set of 3D probe poses and their corresponding ASMs/labels, an ASM-pose mapping model may be created. In some embodiments, the AS-pose mapping model may be a discrete look up table. In some embodiments, a continuous ASM-pose mapping model may be learned, via machine learning, based on the set of 3D poses and their corresponding ASMs/labels as training data. The ASM-pose mapping model obtained pre-surgery according to the present teaching may then be used during a subsequent in-surgery stage.

During a subsequent LUS procedure, a 2D ultrasound image may be acquired by an ultrasound probe manipulated by, e.g., a user or a robot. Such a 2D ultrasound image may be leveraged to estimate a 3D pose of the ultrasound probe. According to the present teaching, if prior 3D probe pose(s) have previously been estimated, a current 3D probe pose corresponding to the current 2D ultrasound image may be estimated, via sequence-aware 3D probe pose estimation, which predicts a 3D probe pose by extrapolating based on a trajectory obtained by fitting the previously estimated 3D probe poses. In some embodiments, it may be specified that there is an adequate number of previously estimated 3D probe poses to ensure the reliability of the fitted trajectory. When there is no previously estimated 3D probe pose (or not having an adequate number of previous estimates), the current 3D probe pose for the current 2D ultrasound image may be estimated by mapping a ASM/label representation of the 2D ultrasound image, via the ASM-pose mapping model, to one or more candidate 3D probe poses with, e.g., some confidence indication such as probabilities. In some embodiments, the ASM/label representation of the 2D current ultrasound image is created by extracting ASMs for 2D structures and their preliminary labels (e.g., preliminary labels such as a vessel without indicating which type of vessel).

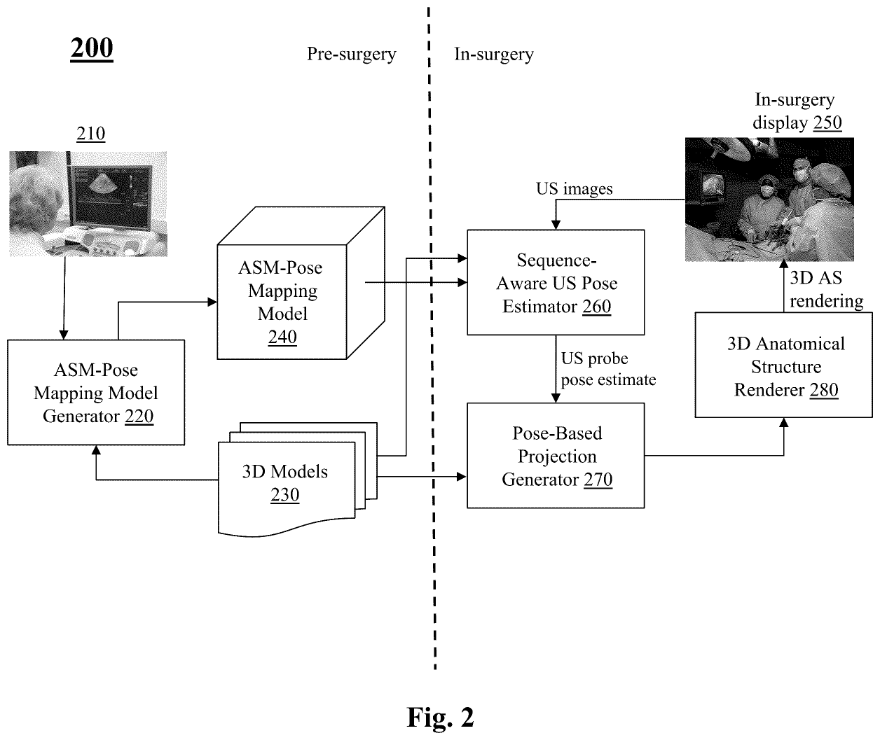

The candidate 3D probe pose estimated for a current 2D ultrasound image may be further optimized. In some embodiments, based on the estimated 3D probe pose (either via sequence-aware prediction or through mapping based on 2D ASM/label representation), a virtual 2D ultrasound image of the 3D model may be obtained which provides ground truth ASMs and associated labels. The ground truth ASMs/labels in the virtual 2D ultrasound image may then be used to update or enhance the ASM labels in the ASM/label representation. Furthermore, the updated ASM/label representation may then be used to re-map to improve the 3D probe pose estimate based on the ASM-pose mapping model to obtain an optimized 3D probe pose estimate for the current 2D ultrasound image. With the optimized 3D probe pose estimate derived according to the present teaching, a corresponding perspective may be determined and used to project 3D anatomical structures from the 3D organ model to offer a 3D view of the anatomical structures as observed in the 2D ultrasound image to assist a surgeon to see what correspond to the 2D information in the 2D ultrasound image. Details of the pre-surgical and in-surgery operations to achieve estimation of a 3D ultrasound probe pose and visualization of 3D anatomical structures determined based on the estimated 3D probe pose are provided herein with references to FIGS. 2-8B.

FIG. 2 depicts an exemplary high level system diagram of a 3D USPP based AS visualization framework 200, in accordance with an embodiment of the present teaching. Operations of this framework has two portions, one being a pre-surgery portion and the other being an in-surgery portion. The pre-surgery portion includes an ASM-pose mapping model generator 220 that interacts with a user 210 to receive a plurality of user specified probe poses with respect to 3D models 230 for a target organ. Based on the user specified probe poses, the ASM-pose mapping model generator 220 creates an ASM-pose mapping model 240. In some embodiments, the ASM-pose mapping model 240 is obtained via machine learning based on training data, generated based on the user specified probe poses.

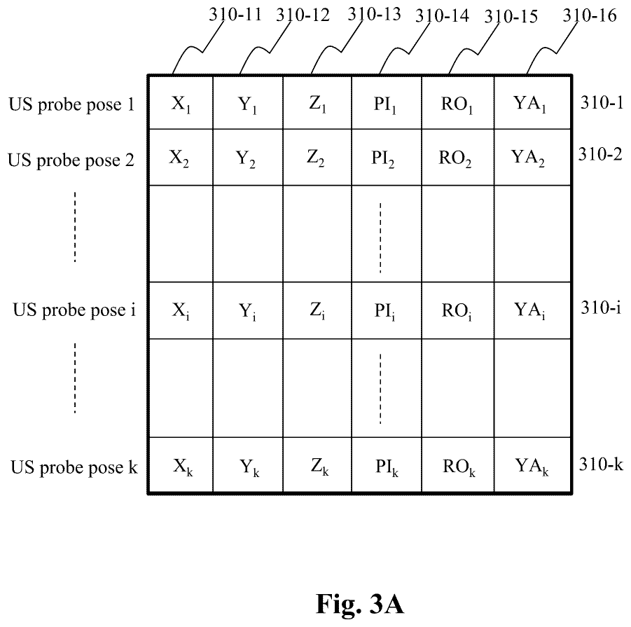

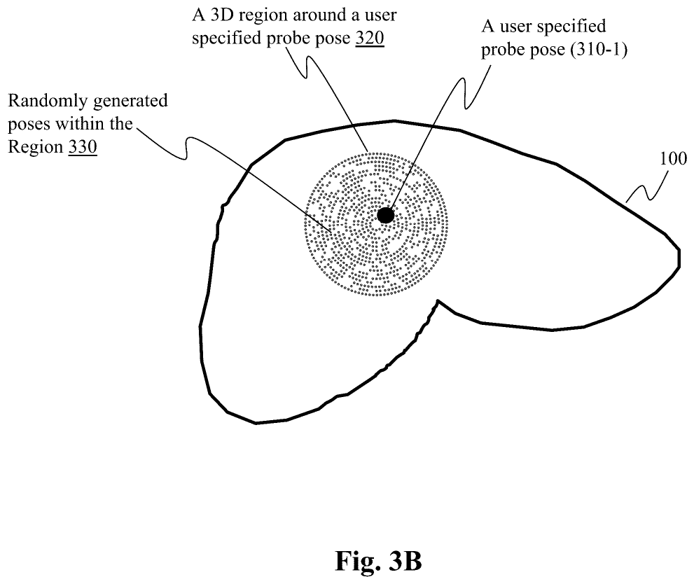

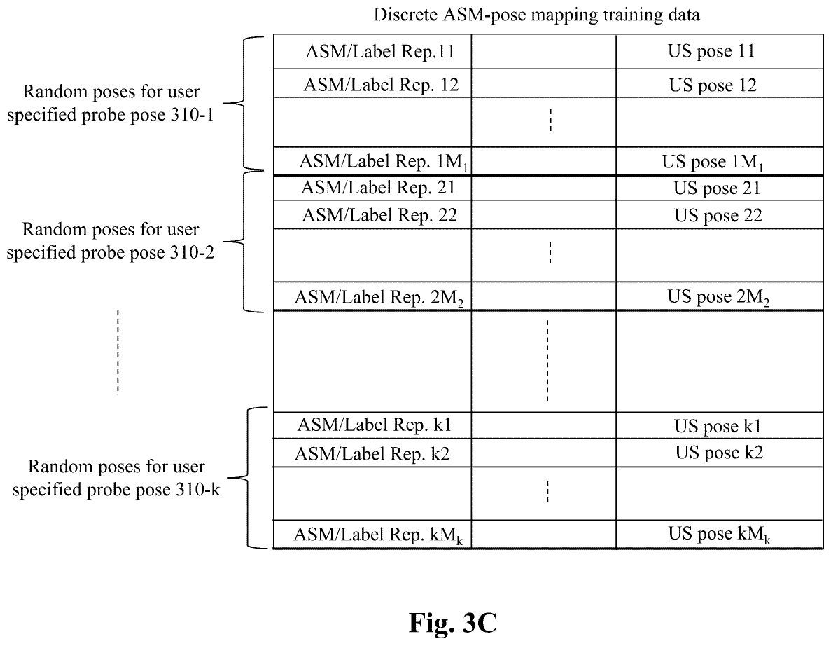

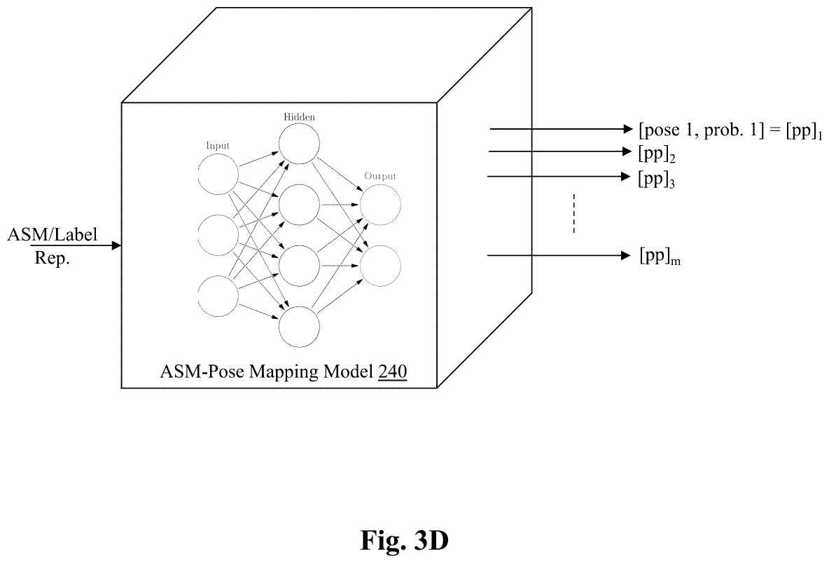

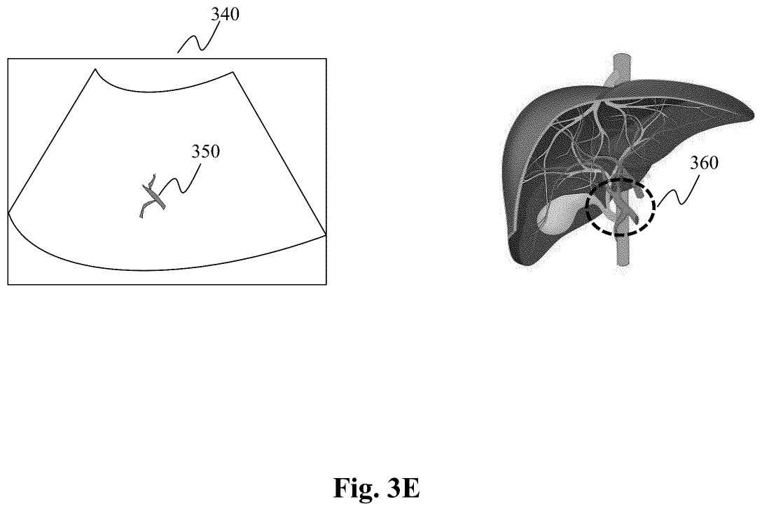

The in-surgery portion comprises a sequence-aware US pose estimator 260, a pose-based projection generator 270, and a 3D anatomical structure renderer 280. During a LUS procedure, 2D ultrasound images may be acquired by a ultrasound probe and the 2D ultrasound (US) images are displayed on an in-surgery display 250. The acquired 2D US images are provided to the sequence-aware US pose estimator 260 for estimating the 3D US probe pose based on the ASM-pose mapping model 240. The estimated 3D US probe pose may then be used by the pose-based projection generator 270, that may generate a 3D projection of the 3D model 230 for a relevant target organ according to a perspective determined based on the estimated 3D US probe pose. The 3D projection of the 3D model includes anatomical structures aligns with the 2D structures as observed in the 2D US images. Such aligned 3D projection is then rendered by the 3D anatomical structure renderer 280 on the display 250 to assist the users in the surgery room to carry out the operation. Details related to the ASM-pose mapping model generator 220 and the sequence-aware US pose estimator 260 are provided with reference to FIGS. 3A-8B.

Before discussing the details associated with the processes associated with the pre-surgery and in-surgery portions of the framework 200, basis for creating the ASM-pose mapping model 240 is discussed herein. To obtain the ASM-pose mapping model 240, the 3D models 230 for the target organ may be visualized at the pre-surgery stage to facilitate a user to select any 3D location as a virtual probe pose. Each user specified probe poses may define a perspective from which to view the 3D models and represented by parameters in six degrees of freedom. FIG. 3A illustrates exemplary K user specified 3D US probe poses and representation thereof, including, e.g., US probe pose 1 310-1, US probe pose 2 310-2, . . . , US probe pose i 310-i, . . . , and US probe pose k 310-k, each represented by parameters in respective 6 degrees of freedom, i.e., 3D coordinate in a 3D coordinate system defined in terms of X (310-11), Y (310-12), and Z (310-13), as well as pitch (PI) 310-14, roll (RO) 310-15, and yaw (YA) 310-16.

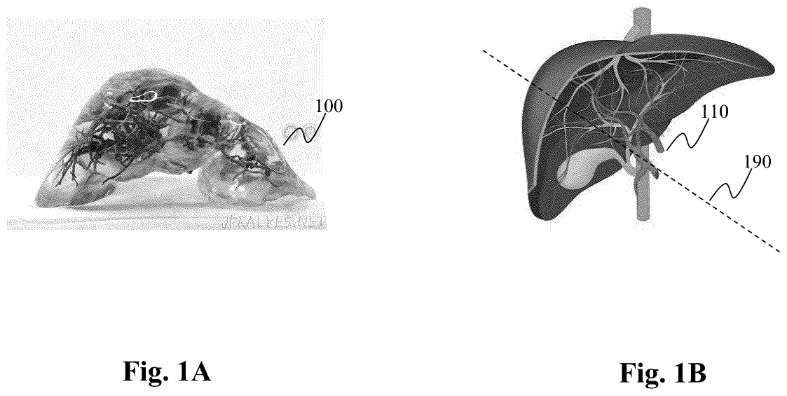

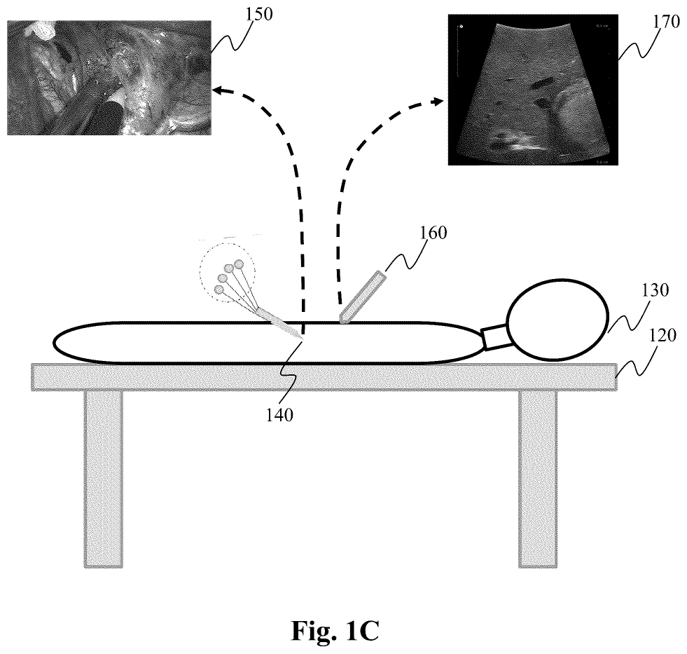

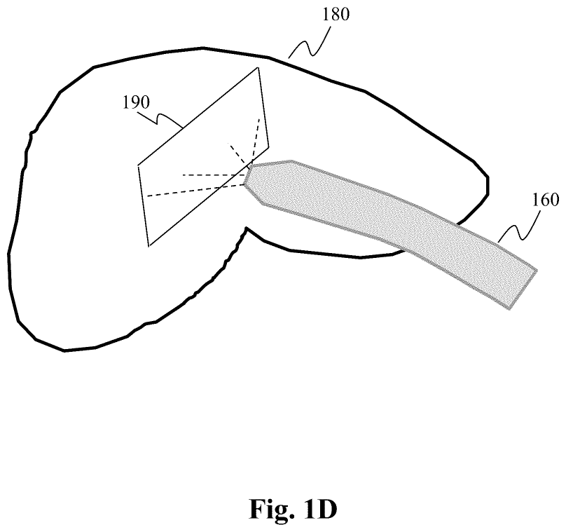

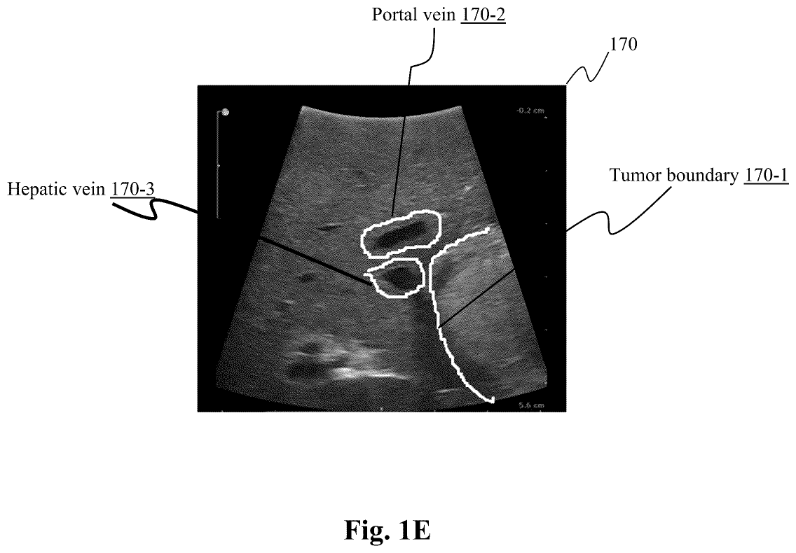

Each of the user specified probe poses may be used to determine a perspective that may be used to determine a slice in the 3D models 230 that corresponds to what a US probe located at the pose sees. One example is illustrated in FIG. 1D, where an ultrasound probe 160 positioned at a particular pose yields a view formed by a slice 190 of the 3D organ 180, wherein the slice is determined based on the operational parameters associated with the US probe (e.g., the depth of the sound wave). That is, each user specified probe pose defines a slice 190 of the 3D organ models 230 and this slice may be used to generate a 2D virtual US image based on the 3D models 230. For each of such 2D virtual US images, anatomical structures as shown in the 2D virtual US image are well defined so that a corresponding ASM for each of the anatomical structures may be generated. In addition, each of the ASMs detected from a 2D virtual US image has a ground truth label made available by the 3D models 230. Thus, there is a correspondence between ASM/label (from the 2D virtual US image) and a 3D probe pose that yields the 2D virtual US image. Such correspondences form a mapping from a ASM/label representation of each 2D virtual US image to a 3D probe pose.

In some embodiments, to construct the ASM-pose mapping model 240, the user specified 3D probe poses may be used as seeds to derive additional 3D probe poses which may then be used to generate more ASM/label—3D probe pose mappings. In some embodiments, the additional mappings may be created in the following way. With respect to each user specified probe pose, a series of probe poses may be randomly generated in a pre-determined vicinity around the user specified pose. For example, based on each user specified probe pose, 10,000 probe poses may be randomly generated within a sphere centered at the user specified probe pose and with a certain radius. FIG. 3B illustrates an example, where for user specified probe pose 1 310-1, a 3D region 320 may be defined and a first set of random poses 330 may be generated within the 3D region 320. Similarly, for user specified probe pose 2 310-2, a second set of random poses may be generated, . . . , for user specified probe pose k 310-k, a kth set of random poses may be generated.

In this manner, many 3D probe poses may be created automatically. Corresponding mappings from ASM/label representations (of 2D virtual US images) to these 3D probe poses may be accordingly generated as discussed herein. That is, for each such generated additional probe pose, a 2D virtual US image may be obtained via the 3D models and an ASM/label representation may be obtained, which corresponds to the probe pose. In this way, many more ASM-pose mappings may be created as shown in FIG. 3C, e.g., ASM/Label Rep. ij-->US pose ij, where 1,1<i,j<k, Mₖ. In this example, 1,1<1, M₁, corresponding to probe poses generated based on the first user specified probe pose 310-1, 2,1<1,M₂, corresponding to probe poses generated based on the second user specified probe pose 310-2, . . . , etc. Although additional probe poses generated with respect to each user specified probe pose is in a random manner within a defined vicinity, it is disclosed herein merely as an illustration rather than as a limitation. Other schemes may be used to generate additional probe poses based on user specified probe poses may also be used.

This process produces a discrete set of ASM-pose mappings as shown in FIG. 3C. In some embodiments, such discrete set of ASM-pose mappings may be used as a discrete ASM-pose mapping model 240. In some embodiments, these discrete ASM-pose mappings may be used as training data, which may be used for training, e.g., via machine learning, a continuous ASM-pose mapping model 240. A continuous ASM-pose mapping model 240 trained in this manner is capable of extrapolating the discrete training data during learning to achieve a much finer resolution. FIG. 3D illustrates an exemplary neural network trained as a continuous ASM-pose mapping model 240. With this mapping model, when an ASM/label rep. obtained from a 2D ultrasound image is provided as input to the ASM-pose mapping model 240, it may output a number of candidate pose estimates, [pp]ᵢ, each of which may include an estimated 3D pose and a metric to represent, e.g., a confidence in the estimate, i.e., [pose i, prob. i].

It may be noted that as each 2D virtual US image includes only certain anatomical structures visible from a perspective determined based on a 3D probe pose, the ASM/Label representation generate therefrom simulates what is seen by an ultrasound probe in that perspective during a surgery. FIG. 3E shows an exemplary 2D US image (either virtual or actual) 340 with an ASM 350 extracted therefrom, which may correspond to some vessels 360 in a vessel tree modeled in a 3D model partially visible in the 2D US image. The ASM-pos mapping model 240 is trained for being able to map a given ASM/label rep. to a 3D probe pose, i.e., based on what is seen in 2D US image to predict what is the 3D probe pose that allows a user to see what is captured in the 2D US image.

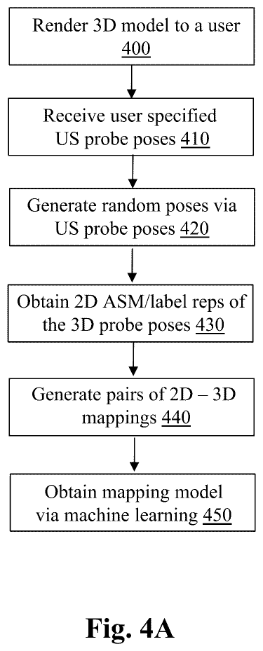

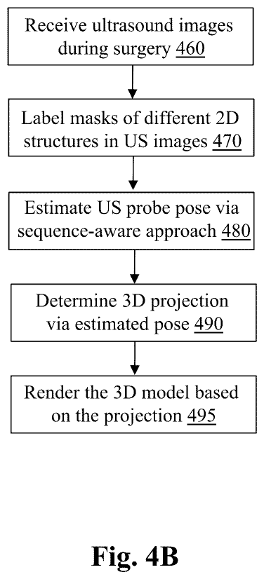

FIG. 4A is a flowchart of an exemplary pre-surgery process of the 3D USPP based AS visualization framework 200 for generating the ASM-pose mapping model 240, in accordance with an embodiment of the present teaching. To allow a user to choose user specified probe poses, the ASM-pose mapping model generator 220 may render, at 400, the 3D models 230 for a target organ to provide a visualization to the user. When information related to a user specified probe pose is received at 410, random probe poses may be generated, at 420, according to the specified 3D probe pose according to some generation scheme as disclosed herein. Based on the specified and additionally generated random probe poses, their ASM/label representations may be generated at 430. The corresponding pairs of ASM/label representation and 3D probe poses may then be generated at 440 and such mappings are then used to obtain, at 450, the ASM-pose mapping model 240. As discussed herein, in some embodiments, the discrete mappings may be used as a discrete mapping model via look-up table operations. In some embodiments, the discrete mappings may be utilized as training data for machine learning a continuous ASM-pose mapping model.

FIG. 4B is a flowchart of an exemplary in-surgery process of the 3D USPP based AS visualization framework 200 for visualizing 3D anatomical structures based on the ASM-pose mapping model 240, in accordance with an embodiment of the present teaching. During a LUS procedure, an ultrasound probe may be deployed to acquire actual ultrasound images. When the sequence-aware US pose estimator 260 receives, at 460, such ultrasound images, it analyzes a 2D US image to identify 2D structures, obtain corresponding ASMs with estimated labels at 470. As the 2D US images are acquired from the procedure, such actual US images may be noisy and poor quality. Given that, the ASMs extracted therefrom as well as the estimated labels may be preliminary. For instance, some extracted 2D structures may be labeled as vessels and some may be labeled as nodules. However, it may not be able to distinguish the type of each vessel and accordingly assign a label corresponding to a specific type of vessel. As will be discussed below, some of the initially estimated preliminary labels may be refined or enhanced according to the present teaching.

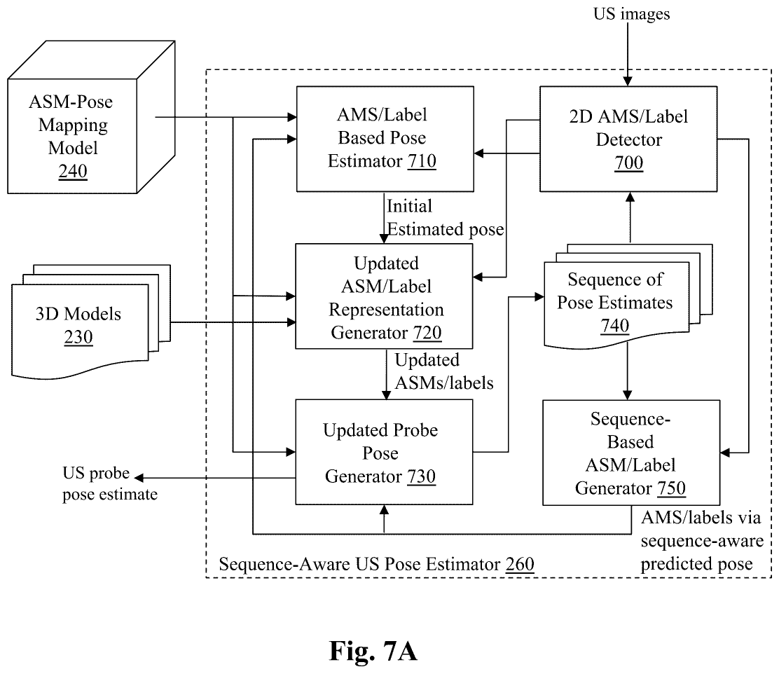

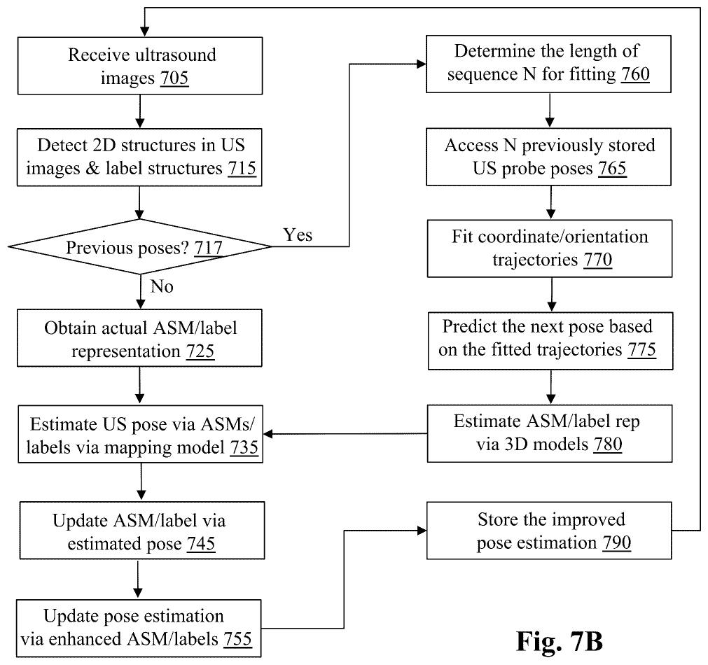

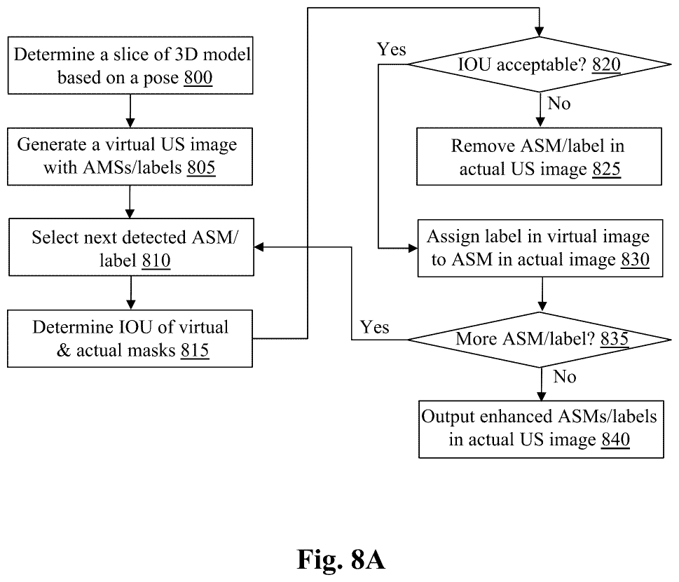

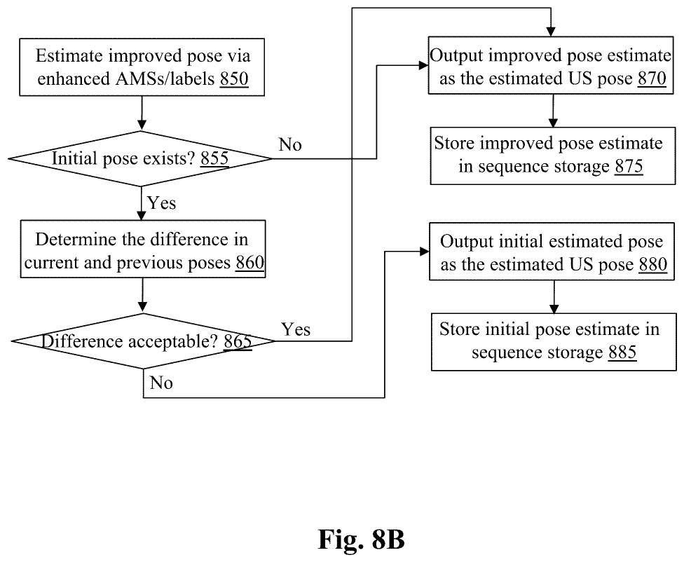

Based on the extracted ASMs and labels, an ASM/label representation for each 2D US image may be obtained and used to estimate, at 480, a 3D pose of the ultrasound probe based on a sequence-aware approach according to the present teaching in accordance with the ASM-pose mapping model 240. Details associated with the sequence-aware estimation approach will be provided with reference to FIGS. 7A-8B. Such an estimated 3D probe pose may then be provided to the pose-based projection generator 270 (see FIG. 2) to determine, at 490, a 3D projection of the 3D models 230 based on a perspective corresponding to the 3D probe pose. The 3D anatomical structure renderer 280 may then render, at 495, the generated projection by visualizing relevant anatomical structures on display 250 in alignment with the 2D structures observable in the 2D US images.

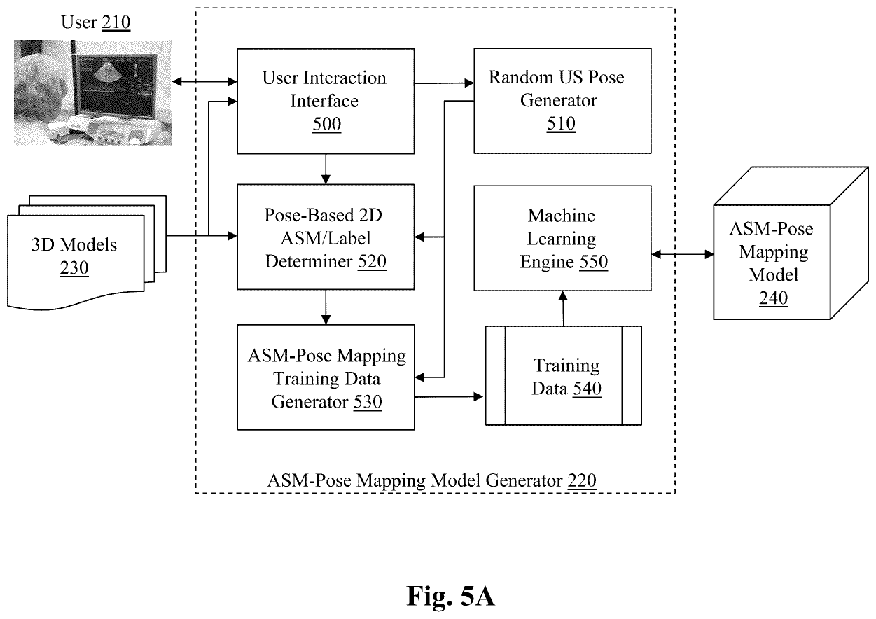

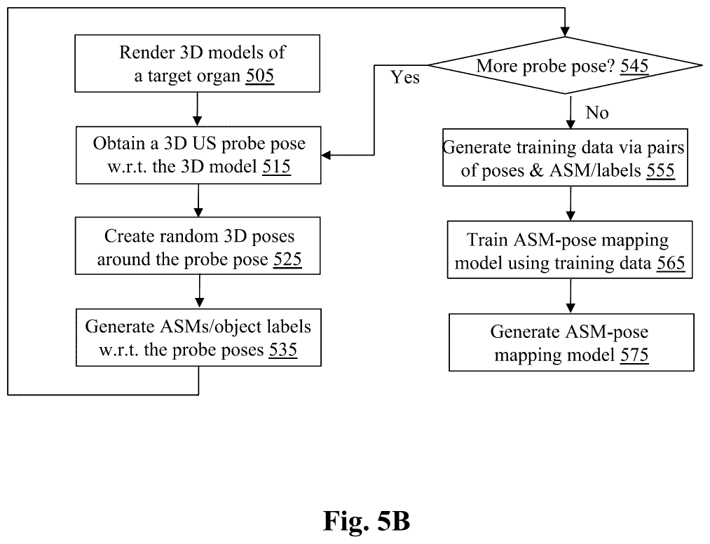

Below, details of the ASM-pose mapping model generator 220 and the sequence-aware US pose estimator 260 are disclosed. FIG. 5A depicts an exemplary high level system diagram of the ASM-pose mapping model generator 220, in accordance with an embodiment of the present teaching. As discussed herein, the ASM-pose mapping model 240 is for mapping an ASM/label representation extracted from a 2D image, which is either via a table look-up operation if the mapping model is discrete or by providing the ASM/label representation to a continuous mapping model to receive its output with estimated candidate 3D probe pose(s). The illustrated embodiment, the ASM-pose mapping model generator 220 is directed to a mode of operation to generate a continuous ASM-pose mapping model 240. With this illustration, the ASM-pose mapping model generator 220 comprises a user interaction interface 500, a random US pose generator 510, a pose based 2D ASM/label determiner 520, an ASM-pose mapping training data generator 530, and a machine learning engine 550.

FIG. 5B is a flowchart of an exemplary process of the ASM-pose mapping model generator 220, in accordance with an embodiment of the present teaching. In operation, the user interaction interface 500 renders, at 505, the 3D models 240 of a target organ to a user. As discussed herein, it is for facilitating the user to define user specified probe poses. In communicating with the user, when the user interaction interface 500 obtains, at 515, a user specified 3D probe pose defined with respect to the 3D models, the random US pose generator 510 generates, at 525, additional 3D probe poses with respect to the user specified probe pose. For the user specified and randomly generated probe poses, the pose-based 2D ASM/label determiner 520 generates, at 535, ASM/label representations for such probe poses. As discussed herein, to do so, with respect to each 3D probe pose, a corresponding 2D virtual US image may be determined based on a slice of the 3D models 240 determined according to the 3D probe pose. Based on the 2D virtual US image, ASMs and corresponding labels may be identified and used to form an ASM/label representation of the 2D virtual US image. Each of the ASM/label representations and its associated 3D probe pose (either user specified or randomly generated) form a pair or mapping, i.e., ASM/label rep.-->pose.

The process of generating discrete mappings for each 3D probe pose (either user specified or randomly generated) may continue until the user completes selecting user specified probe poses, determined at 545. With such generated N discrete mappings created based on 3D models (e.g., N=k×M, where k is the number of user specified probe poses and M is the number of probe poses generated with respect to each user specified probe pose), the ASM-pose mapping training data generator 530 generates, at 555, training data 550, which is then used by the machine learning engine 550 to perform training, at 565, to derive, at 575, the trained ASM-pose mapping model 240. As discussed herein, through machine learning, the trained ASM-pose mapping model 240 learns the relationship between 2D ASM/label representations of the 3D anatomical structures (e.g., ASM/labels) at any 3D probe pose within the range it is being trained on. That is, using the N discrete ASM-pose mappings for training derives a trained ASM-pose mapping model 240 that can estimate a 3D probe pose given what is observed in a 2D US image from any perspective.

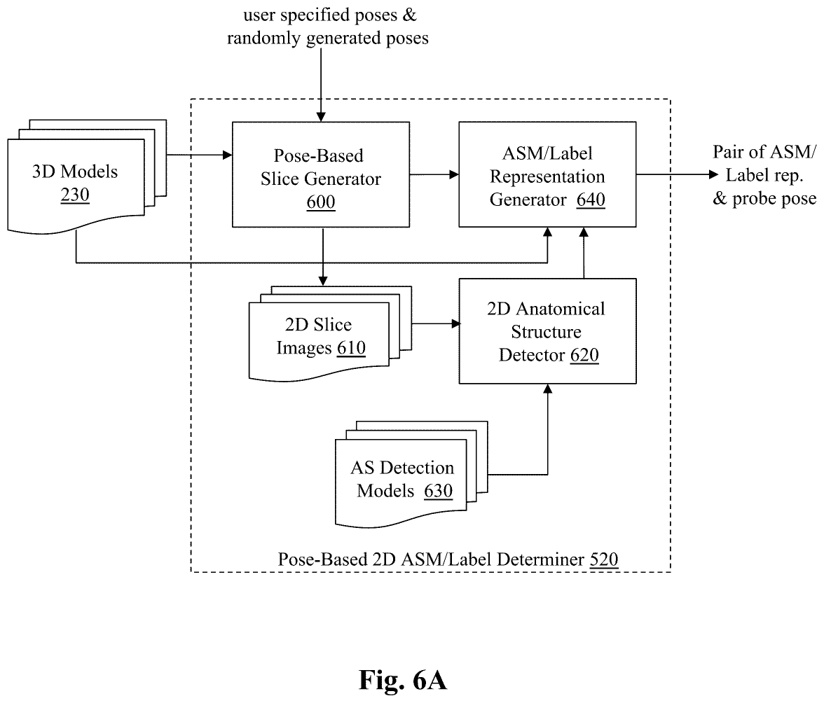

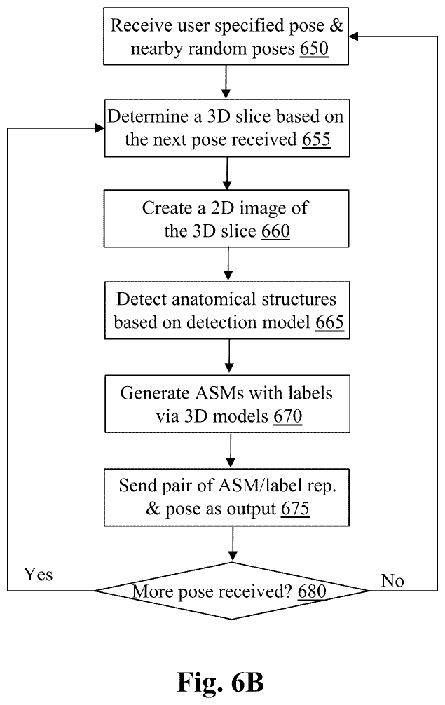

FIG. 6A depicts an exemplary high level system diagram of the pose-based 2D ASM/label determiner 520, in accordance with an embodiment of the present teaching. As discussed herein, the pose-based ASM/label determiner 520 is to translate each given probe pose (either user specified or randomly generated) into an ASM/label representation, i.e., taking each input pose as an input and producing an ASM/label representation as output. This enables the pairing of each ASM/label representation with a corresponding pose. In some embodiments, the pose-based ASM/label determiner 520 comprises a pose-based slice generator 600, a 2D anatomical structure detector 620, and an ASM/label representation generator 640.

The pose-based slice generator 600 is provided to simulate the process of acquiring an ultrasound image by an ultrasound probe at a given 3D probe pose. Given an input probe pose, the pose-based slice generator 600 determines a plane in the 3D models 230 sliced according to a perspective determined based on the probe pose with respect to the 3D models. Based on the slice, the pose-based slice generator 600 generates a 2D virtual US image with anatomical structures therein as what would be sensed by an ultrasound probe from the perspective at the probe pose. The 2D virtual US images created based on input probe poses may be stored in 610. The 2D anatomical structure detector 620 is provided for detecting anatomical structures in each of the 2D virtual US image. Such anatomical structures may include tumor(s) and blood vessels. The detection may be carried out based on some relevant AS detection models 630. Based on the detected anatomical structures, the ASM/Label representation generator 640 is provided to generate corresponding ASM/label representations by, e.g., creating binary ASM for each detected anatomical structure and assigning a label thereto. As each 2D virtual US image is generated based on the 3D models 230, the labels for the detected ASMs may be readily retrieved from the 3D models in creating an ASM/label representation for each 2D virtual US image.

FIG. 6B is a flowchart of an exemplary process of the pose-based 2D ASM/label determiner 520, in accordance with an embodiment of the present teaching. In operation, when the pose-based slice generator 600 receives, at 650, input probe poses (including both user specified and additional poses generated), it determines, at 655, for each of the input poses, a 3D slice and creates, at 660, a 2D virtual US image based on the 3D slice. For each 2D virtual US image so created, the 2D anatomical structure detector 620 detects, at 665, anatomical structures appearing in the 2D virtual US image. The anatomical structures in a 2D virtual US image ma then be used by the ASM/label representation generator 640 to generate, at 670, an ASM/label representation corresponding to the input probe pose and sends, at 675, the ASM/label representation and the corresponding probe pose as a pair as an output. This process of generating 2D virtual US images, detecting anatomical structures from each 2D virtual US image, and creating ASM-pose pairs continues until, determined at 680, all ASM-pose mappings are generated for the input probe poses.

As discussed herein, the pre-surgery part of the 3D USPP based AS visualization framework 200 is for creating user specified probe poses and generating the ASM-pose mapping model 240 based on user specified probe poses, which is then utilized in the in-surgery stage to estimate 3D US probe poses to facilitate registered visualization of anatomical structures corresponding to what is captured in 2D ultrasound images. As disclosed herein, during the surgery, the sequence-aware US pose estimator 260 estimates, based on the ASM-pose mapping model 240, the 3D probe pose corresponding to the ultrasound images acquired during a LUS procedure.

FIG. 7A depicts an exemplary high level system diagram of the sequence-aware US pose estimator 260, in accordance with an embodiment of the present teaching. As discussed herein, the sequence-aware US pose estimator 260 takes an ultrasound image acquired by the ultrasound probe 160 during a LUS procedure as input and produces an estimated 3D ultrasound probe pose as output. In this illustrated embodiment, the sequence-aware US pose estimator 260 comprises a 2D AMS/label detector 700, a sequence-based ASM/label generator 750, an ASM/label-based pose estimator 710, an updated ASM/label representation generator 720, and an updated probe pose generator 730.

As discussed herein, when an ultrasound image is received, there may be alternative ways to obtain an ASM/label representation of the US image. For example, ASMs and corresponding labels may be detected and estimated from the input US image. The 2D AMS/label detector 700 is provided for extracting ASMs and estimating corresponding labels from the input US image. Another alternative is to obtain ASMs/labels corresponding to the input US image via a sequence-aware approach by predicting a probe pose based on a sequence of previously estimated probe poses. The sequence-based ASM/label generator 750 is provided for estimating the ASM/label representation based on a historic sequence of probe poses. With the ASM/label representation, either detected based on the input US image or predicted based on a sequence of previous probe poses, the AMS/label based pose estimator 710 is provided for mapping the ASM/label representation to a 3D probe pose based on the ASM-pose mapping model 240 to produce an initially estimated probe pose of the ultrasound probe.

The sequence-aware US pose estimator 260 according to the present teaching in this illustrated embodiment is provided to further optimize the 3D probe pose estimated by the AMS/label based pose estimator 710. The updated ASM/label representation generator 720 is provided for enhancing the ASM/label representation created by either 700 or by 750 based on the initially estimated probe pose. The updated ASM/label representation generator is provided to utilize the initially estimated probe pose (from the AMS/label based pose estimator 710) to access ground truth labels for anatomical structures observable in the 3D models 230 from a perspective determined based on the initially estimated probe pose and to generate an updated ASM/label representation based on such ground truth labels. The updated probe pose generator 730 may then in turn utilize the updated ASM/label representation to further improve the probe pose estimation by using the updated ASM/label representation to map to an updated probe pose based on the ASM-pose mapping model 240. That is, the 3D models 230 and the AMS-pose mapping model 240 are leveraged to enhance the ASM/label representation to obtain an optimized probe pose estimate. The optimized probe pose for each input US image may then be output as the estimated 3D probe pose for the input US image. To facilitate sequence-aware estimation of the initial ASM/label representation for the next input US image, the estimated 3D probe pose is also stored in the sequence of pose estimate storage 740.

FIG. 7B is a flowchart of an exemplary process of the sequence-aware US pose estimator 260, in accordance with an embodiment of the present teaching. When an input US image is received at 705, 2D structures are detected therefrom and anatomical structure masks (ASMs) and corresponding labels for such 2D structures are estimated at 715. The 2D structures may be used to generate ASMs with labels preliminarily estimated. As discussed herein, the labels assigned to the detected ASMs may correspond to weak labels such as a label for organ and a label for vessels. It is then determined, at 717, whether there is adequate number of previously estimated poses. If there is no previously estimated probe poses or there is not an adequate number of prior poses available, the 2D AMS/label detector 700 obtains, at 725, an ASM/label representation for the input US image based on the ASM(s) and label(s) detected/estimated from the input US image. As discussed herein, as an ultrasound image is generally noisy and incomplete, an ASM/label representation obtained therefrom is less reliable and inaccurate. Thus, the present teaching discloses another alternative to obtain an ASM/label representation for an input US image based on a sequence of previously estimated probe poses by leveraging the 3D models for a target organ.

When there is an adequate number of prior probe poses in 740, determined at 717, the ASM/label representation for the input US image may be predicted via a sequence-aware approach. In some embodiments, the adequate number of previously estimated poses may be configured in such a way to ensure that the ASM/label representation to be predicted for the input US image may be reliably obtained based on the sequence of prior poses. When the sequence-based ASM/label generator 750 is invoked to predict ASM/label representation for the input US image, it may first determine, at 760, a length of representing the number of prior probe poses to be used for fitting. Then accordingly N prior poses may be retrieved at 765 from storage 740. As discussed herein, each 3D pose is represented by parameters in six degrees of freedom, corresponding to respectively a 3D coordinate and a 3D orientation (represented by roll, pitch, and yaw). In some embodiments, the fitting may be separately carried out, at 770, with respect to the N prior probe poses. Such fittings yield two trajectories, one corresponding to the trajectory of 3D coordinates and one corresponding to the trajectory of 3D orientations.

These two trajectories may then be used to predict, at 775, via, e.g., extrapolation, the next 3D probe pose, including the next 3D coordinate predicted by extrapolating the trajectory for 3D coordinates and the next 3D orientation predicted by extrapolating from the trajectory for 3D orientations. Based on the predicted 3D probe pose, a virtual ultrasound image may be created based on the 3D models 230, which may then be processed to determine ASMs and labels (ground truth labels from the 3D models) and generate, at 780, a predicted ASM/label representation for the input US image. The ASM/label representation predicted using sequence-aware approach may be more reliable especially when there are an adequate number of prior poses. In some embodiments, the adequate number of previously estimated poses may be configured in such a way to ensure that prediction of ASM/label representation for the input US image based on the sequence of prior poses is reliable.

With the ASM/label representation, either estimated from the input US image at 725 or predicted at 780 based on a sequence of prior poses, the ASM/label based pose estimator 710 estimates, at 735, an initial 3D estimated pose for the input US image based on the ASM-pose mapping model 240. As discussed herein, this initial estimated pose may further be optimized. In this illustrated embodiment, the updated ASM/label representation generator 720 may be invoked to update, at 745, the ASM/label representation by leveraging the initial estimated pose. For example, using the initial estimated pose, a virtual ultrasound image may be generated via the 3D models 230 and used to obtain the updated ASM/label representation (by extracting ASMs and retrieving ground truth labels from the 3D models). Furthermore, the updated probe pose generator 730 may further be invoked to generate, at 755, an updated pose estimation by mapping the updated ASM/label representation to an updated probe pose via the ASM-pose mapping model 240. Details on these further optimization operations are provided with reference to FIGS. 8A-8B. The updated probe pose estimate may then be output and stored, at 790, in 740 to the sequence of poses to facilitate sequence aware ASM/label representation prediction.

As discussed herein, the ASM/label representation for the input US image used for obtaining the initial estimated pose may be enhanced via the initial estimated pose by leveraging the 3D models 230 for the target organ. FIG. 8A is a flowchart of an exemplary process for enhancing ASM/label representation based on the initial estimated pose, in accordance with an embodiment of the present teaching. With the initial estimated pose, the updated ASM/label representation generator 720 may determine, at 800, a slice of the 3D models 230 according to a perspective determined by the initial estimated pose. Such a slice is also determined based on operational parameters (e.g., depth) of the ultrasound probe at issue. Based on the slice, a 2D virtual US image is generated at 805, that includes information related to some anatomical structure(s) and associated with ground truth label(s), based on which ASM(s) and label(s) thereto may be accordingly derived.

For each virtual ASM in the 2D virtual US image, a corresponding actual ASM (in the AMS/label representation associated with the input US image) is selected at 810 and an intersection of union (IOU) between the virtual and actual ASMs may be determined at 815. In some embodiments, the IOU may be defined to represent an overlap between the two ASMs. If the IOU is not acceptable, determined at 820, according to some defined criterion, the actual ASM associated with the input US image and its estimated label may be removed at 825. For example, when the IOU is low, the reliability of the corresponding actual ASM is low so that it may be removed together with the associated label. If the IOU between the virtual and actual ASM is acceptable, the ground truth label associated with the virtual ASM is used to assign, at 830, the ground truth label as the estimated label for the actual ASM. For example, an estimated label for the actual ASM may be vessel, while the ground truth label for the virtual ASM may be a portal vein, which is more accurate and may be used to enhance the labeling to the actual ASM. The process as described above is repeated for each of the actual ASMs based on the initial estimated pose until, determined at 835, all actual ASMs/labels have been so processed to enhance the labels in the ASM/label representation for the input US image. When the process ends, the ASM/label representation with enhanced labels so obtained corresponds to an updated ASM/label representation for the input US image and is output at 840.

As discussed herein, the updated ASM/label representation for the input US image (with enhanced labels) may then further be utilized to improve the mapping (from updated ASM/label representation) to obtain an updated estimated probe pose by leveraging the ASM-pose mapping model 240. FIG. 8B is a flowchart of an exemplary process for updating an initial estimated 3D pose based on an updated ASM/label representation, in accordance with an embodiment of the present teaching. When the updated probe pose generator 730 receives an updated ASM/label representation from the updated ASM/label representation generator 720, it utilizes it to estimate, at 850, an updated estimated probe pose via the ASM-pose mapping model 240. In some embodiments, it is also possible that the ASM/label representation is from the sequence-based ASM/label generator 750 (i.e., predicted in a sequence-aware manner) which may also be used to estimate a 3D probe pose.

If the estimated 3D probe pose corresponds to an improved estimate obtained based on the initial estimated pose, determined at 855, then the process proceeds to determine, at 860, a difference between the initial and the improved estimated poses. If the different is acceptable, determined at 865, then the improved estimated pose is to replace the initial estimated pose as the estimated pose and is output at 870. The output estimated pose may also be stored, at 875, in the storage 740 as an addition to a sequence of historic estimated poses. If the estimated pose obtained at step 850 is based on predicted ASM/label representation from 750, there is no initial estimated pose because the pose is estimated based on the ASMs/labels predicted according to the prior poses in the sequence 740. When no initial estimated probe pose exists (e.g., when the input ASM/label representation is from the sequence-based ASM/label generator 750), the estimated probe pose is output at 870 and is stored, at 875, in the storage 740 as the new pose to a sequence of historic estimated probe pose.

In the above disclosed processes, the 3D probe pose estimate is optimized in different situations to improve its accuracy and reliability so that the estimated 3D probe pose may be used, by the pose-based projection generator 270, to register the 3D models 230 with the US image to generate a projection and by the 3D anatomical structure renderer 280 to visualize 3D anatomical structures corresponding to what is visible in the 2D US images in an aligned manner to provide effective assistance to the medical personnel.

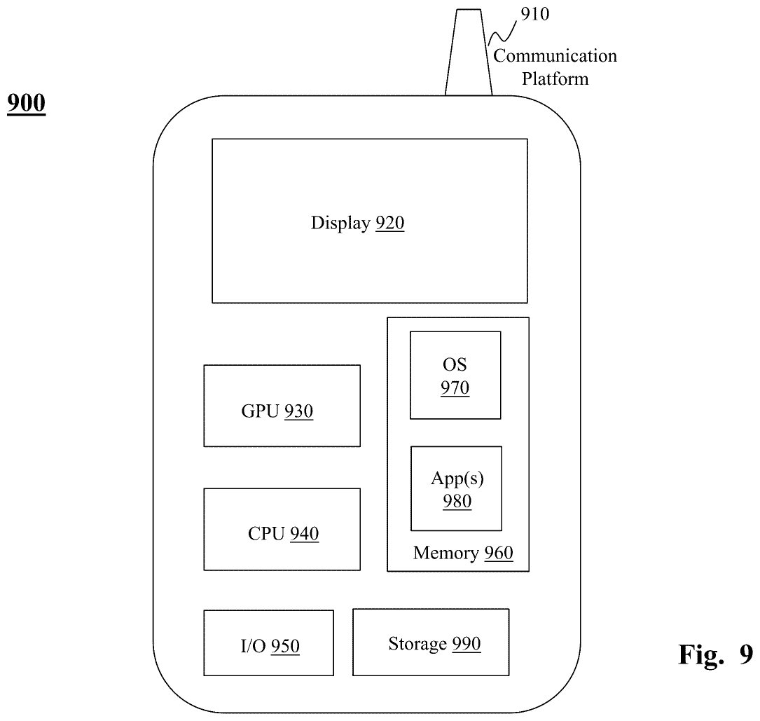

FIG. 9 is an illustrative diagram of an exemplary mobile device architecture that may be used to realize a specialized system implementing the present teaching in accordance with various embodiments. In this example, the user device on which the present teaching may be implemented corresponds to a mobile device 900, including, but not limited to, a smart phone, a tablet, a music player, a handled gaming console, a global positioning system (GPS) receiver, and a wearable computing device, or in any other form factor. Mobile device 900 may include one or more central processing units (“CPUs”) 940, one or more graphic processing units (“GPUs”) 930, a display 920, a memory 960, a communication platform 910, such as a wireless communication module, storage 990, and one or more input/output (I/O) devices 950. Any other suitable component, including but not limited to a system bus or a controller (not shown), may also be included in the mobile device 900. As shown in FIG. 8, a mobile operating system 970 (e.g., iOS, Android, Windows Phone, etc.), and one or more applications 980 may be loaded into memory 960 from storage 990 in order to be executed by the CPU 940. The applications 980 may include a user interface or any other suitable mobile apps for information analytics and management according to the present teaching on, at least partially, the mobile device 900. User interactions, if any, may be achieved via the I/O devices 950 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result the drawings should be self-explanatory.

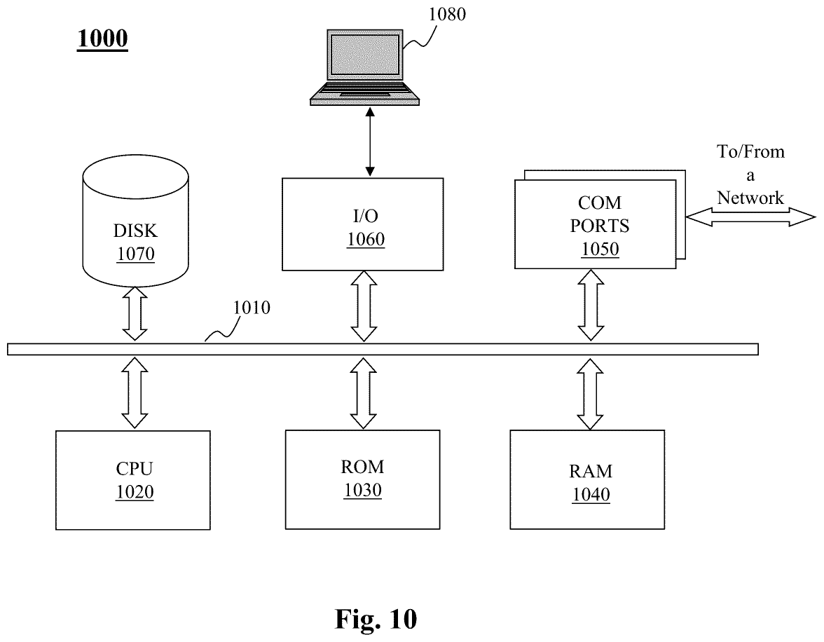

FIG. 10 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special purpose computer. Both can be used to implement a specialized system for the present teaching. This computer 1000 may be used to implement any component or aspect of the framework as disclosed herein. For example, the information analytical and management method and system as disclosed herein may be implemented on a computer such as computer 1000, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 1000, for example, includes COM ports 1050 connected to and from a network connected thereto to facilitate data communications. Computer 1000 also includes a central processing unit (CPU) 1020, in the form of one or more processors, for executing program instructions. The exemplary computer platform includes an internal communication bus 1010, program storage and data storage of different forms (e.g., disk 1070, read only memory (ROM) 1030, or random-access memory (RAM) 1040), for various data files to be processed and/or communicated by computer 1000, as well as possibly program instructions to be executed by CPU 1020. Computer 1000 also includes an I/O component 1060, supporting input/output flows between the computer and other components therein such as user interface elements 1080. Computer 1000 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine-readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as a main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method, comprising:
receiving an ultrasound image acquired by an ultrasound probe deployed at a three-dimensional (3D) probe pose during a medical procedure involving a target organ;
detecting each anatomical structure from the ultrasound image and an anatomical structure mask (ASM) thereof;
estimating a label for each ASM to generate an ASM/label pair;
if a sequence of previous 3D probe poses exists, representing prior 3D probe poses of the ultrasound probe,
predicting the 3D probe pose based on the sequence of previous 3D probe poses;
creating a virtual ultrasound image based on the predicted 3D probe pose,
obtaining an ASM/label representation of the virtual ultrasound image based on at least one ASM/label pair obtained from the virtual ultrasound image; and
if the sequence of previous 3D probe poses does not exist,
obtaining an ASM/label representation based on at least one ASM/label pair obtained from the ultrasound image, and
estimating the 3D probe pose based on the ASM/label representation of the ultrasound image via an ASM-pose mapping model that maps the ASM/label representation to a 3D probe pose.

2. The method of claim 1, wherein the step of predicting comprises:
generating a first trajectory of 3D coordinates of the prior 3D probe poses in the sequence;
generating a second trajectory of 3D orientations of the prior 3D probe poses in the sequence;
generating the predicted 3D probe pose based on the first and the second trajectories, wherein
the virtual ultrasound image is created based on a 3D model for the target organ in accordance with the predicted 3D probe pose.

3. The method of claim 2, wherein the step of obtaining the at least one ASM/label pair from a virtual ultrasound image comprises:
identifying each 2D structure in the virtual ultrasound image corresponding to a 3D anatomical structure in the 3D model;
generating a mask for the 2D structure to create a corresponding ASM; and
assigning a label for the 3D anatomical structure retrieved from the 3D model to the ASM to generate the ASM/label pair.

4. The method of claim 1, further comprising updating the ASM/label representation based on the 3D probe pose to generate an updated ASM/label representation for the ultrasound image, wherein the updating comprises:
creating a new virtual ultrasound image based on the 3D probe pose in accordance with the 3D model;
extracting one or more virtual ASM/label pairs from the new virtual ultrasound image;
for each ASM/label pair generated based on the ultrasound image,
identifying a corresponding virtual ASM/label pair,
revising the ASM/label pair from the ultrasound image if it satisfies at least one predetermined criterion with respect to the virtual ASM/label pair; and
generating an update ASM/label representation based on the revised ASM/label pair.

5. The method of claim 4, wherein the step of revising according to at least one predetermined criterion comprises:
if an overlap between the ASM in the ASM/label pair and the ASM in the virtual ASM/label pair is not acceptable, removing the ASM/label pair;
if the overlap between the ASM in the ASM/label pair and the ASM in the virtual ASM/label pair is acceptable, replacing the label from the ASM/label pair with the label from the virtual ASM/label pair.

6. The method of claim 4, further comprising updating the 3D probe pose to generate an updated 3D probe pose based on the updated ASM/label representation, wherein the step of updating the 3D probe pose comprises:
obtaining a new 3D probe pose based on the updated ASM/label representation via the ASM-pose mapping model;
if the new 3D probe pose and the 3D probe pose satisfy a configured condition, outputting the new 3D probe pose as the updated 3D probe pose; and
if the new 3D probe pose and the 3D probe pose do not satisfy the configured condition, outputting the 3D probe pose as the updated 3D probe pose.

7. The method of claim 6, wherein the configured condition defines that a deviation between the new 3D probe pose and the 3D probe pose is at an acceptable range.

8. The method of claim 6, further comprising adding the updated 3D probe pose to the sequence of previous 3D probe poses of the ultrasound probe.

9. The method of claim 1, wherein the ASM-pose mapping model is created prior to the medical procedure by:
retrieving a 3D model of the target organ;
generating a plurality of virtual 3D probe poses in connection with an ultrasound probe;
creating a virtual ultrasound image with respect to each of the plurality of virtual 3D probe poses;
obtaining an ASM-pose pairing for each of the plurality of virtual 3D probe poses, where the ASM-pose pairing includes a virtual 3D probe pose and an ASM/label representation for the virtual ultrasound image created with respect to the virtual 3D probe pose; and
establishing the ASM-pose mapping model for mapping an ASM/label representation to a 3D probe pose based on the ASM-pose pairings.

10. The method of claim 9, wherein the step of establishing comprises:
generating training data based on the ASM-pose pairings;
obtaining, via machine learning based on the training data, the ASM-pose mapping model to learn relationships between ASM/label representations and 3D probe poses.

11. A machine-readable and non-transitory medium having information recorded thereon, wherein the information, when read by the machine, causes the machine to perform the following steps:
receiving an ultrasound image acquired by an ultrasound probe deployed at a three-dimensional (3D) probe pose during a medical procedure involving a target organ;
detecting each anatomical structure from the ultrasound image and an anatomical structure mask (ASM) thereof;
estimating a label for each ASM to generate an ASM/label pair;
if a sequence of previous 3D probe poses exists, representing prior 3D probe poses of the ultrasound probe,
predicting the 3D probe pose based on the sequence of previous 3D probe poses;
creating a virtual ultrasound image based on the predicted 3D probe pose,
obtaining an ASM/label representation of the virtual ultrasound image based on at least one ASM/label pair obtained from the virtual ultrasound image; and
if the sequence of previous 3D probe poses does not exist,
obtaining an ASM/label representation based on at least one ASM/label pair obtained from the ultrasound image, and
estimating the 3D probe pose based on the ASM/label representation of the ultrasound image via an ASM-pose mapping model that maps the ASM/label representation to a 3D probe pose.

12. The medium of claim 11, wherein the step of predicting comprises:
generating a first trajectory of 3D coordinates of the prior 3D probe poses in the sequence;
generating a second trajectory of 3D orientations of the prior 3D probe poses in the sequence;
generating the predicted 3D probe pose based on the first and the second trajectories, wherein
the virtual ultrasound image is created based on a 3D model for the target organ in accordance with the predicted 3D probe pose.

13. The medium of claim 12, wherein the step of obtaining the at least one ASM/label pair from a virtual ultrasound image comprises:
identifying each 2D structure in the virtual ultrasound image corresponding to a 3D anatomical structure in the 3D model;
generating a mask for the 2D structure to create a corresponding ASM; and
assigning a label for the 3D anatomical structure retrieved from the 3D model to the ASM to generate the ASM/label pair.

14. The medium of claim 11, wherein the information, when read by the machine, further causes the machine to perform the step of updating the ASM/label representation based on the 3D probe pose to generate an updated ASM/label representation for the ultrasound image, wherein the updating comprises:
creating a new virtual ultrasound image based on the 3D probe pose in accordance with the 3D model;
extracting one or more virtual ASM/label pairs from the new virtual ultrasound image;
for each ASM/label pair generated based on the ultrasound image,
identifying a corresponding virtual ASM/label pair,
revising the ASM/label pair from the ultrasound image if it satisfies at least one predetermined criterion with respect to the virtual ASM/label pair; and
generating an update ASM/label representation based on the revised ASM/label pair.

15. The medium of claim 14, wherein the step of revising according to at least one predetermined criterion comprises:
if an overlap between the ASM in the ASM/label pair and the ASM in the virtual ASM/label pair is not acceptable, removing the ASM/label pair;
if the overlap between the ASM in the ASM/label pair and the ASM in the virtual ASM/label pair is acceptable, replacing the label from the ASM/label pair with the label from the virtual ASM/label pair.

16. The medium of claim 14, wherein the information, when read by the machine, further causes the machine to perform the step of updating the 3D probe pose to generate an updated 3D probe pose based on the updated ASM/label representation, wherein the step of updating the 3D probe pose comprises:
obtaining a new 3D probe pose based on the updated ASM/label representation via the ASM-pose mapping model;
if the new 3D probe pose and the 3D probe pose satisfy a configured condition, outputting the new 3D probe pose as the updated 3D probe pose; and
if the new 3D probe pose and the 3D probe pose do not satisfy the configured condition, outputting the 3D probe pose as the updated 3D probe pose.

17. The medium of claim 16, wherein the configured condition defines that a deviation between the new 3D probe pose and the 3D probe pose is at an acceptable range.

18. The medium of claim 16, wherein the information, when read by the machine, further causes the machine to perform the step of adding the updated 3D probe pose to the sequence of previous 3D probe poses of the ultrasound probe.

19. The medium of claim 11, wherein the ASM-pose mapping model is created prior to the medical procedure by:
retrieving a 3D model of the target organ;
generating a plurality of virtual 3D probe poses in connection with an ultrasound probe;
creating a virtual ultrasound image with respect to each of the plurality of virtual 3D probe poses;
obtaining an ASM-pose pairing for each of the plurality of virtual 3D probe poses, where the ASM-pose pairing includes a virtual 3D probe pose and an ASM/label representation for the virtual ultrasound image created with respect to the virtual 3D probe pose; and
establishing the ASM-pose mapping model for mapping an ASM/label representation to a 3D probe pose based on the ASM-pose pairings.

20. The medium of claim 19, wherein the step of establishing comprises:
generating training data based on the ASM-pose pairings;
obtaining, via machine learning based on the training data, the ASM-pose mapping model to learn relationships between ASM/label representations and 3D probe poses.
